(19) United States
(12) Patent Application Publication
(10) Pub. No.: US 2022/0401265 A1
(43) Pub. Date: Dec. 22, 2022

(54) Welding Hood

(71) Applicant: Richard Nelson Williams, Washington, PA (US)

(72) Inventor: Richard Nelson Williams, Washington, PA (US)

(21) Appl. No.: 17/349,984

(22) Filed: Jun. 17, 2021

(51) Int. Cl. A61F 9/06

(52) U.S. Cl. CPC A61F 9/061; A61F 9/068

(57) ABSTRACT

A welding hood for protecting an operator's face comprising a face protective shield having a rearward side configured for receiving the operator's face and a viewing opening extending through the shield. The viewing opening is located in front of the eyes of the operator while the hood is fixed to the operator's head. An eye box is located adjacent the viewing opening and extends inwardly from the rearward side of the shield to block reflective arc flashes, sunlight, and/or shop-lighting from entering the operator's field of view during welding. The welding hood includes a light mounted to the face protective shield for shining light outward from a front side of the face protective shield, a cooling fan mounted adjacent the rearward side of the shield, and a clear protective member mounted within the eye box and adjacent the operator's eyes.

### BACKGROUND OF THE INVENTION

### Field of the Invention

The present invention is directed to a welding hood, and more particularly, to a welding hood having a fitted eye box, extended side portions, a light, a safety lens, and a fan, resulting in improved safety and comfort.

### Description of Related Art

Welding helmets or hoods are well-known protective gear for welders and have been in use since around the 1930's. One example of a currently available and known welding hood is shown in prior art FIGS. 1-3 of the present application. As can be seen from the prior art figures, many of these known models provide little comfort to the operator, provide little to no blockage of reflective arc flash, sunlight, and/or shop-lighting from entering behind the face shield, and provide little to no blockage of sparks, dirt, and/or debris from getting in the eyes of the operator. These known welding hoods also do not provide additional protection of the operator's ears and/or neck, leaving much of these areas exposed. These known welding hoods also do not include a built in light, a cooling fan, and/or additional eye protection, in addition to the lens in the viewing window.

The welding hood of the present invention overcomes the deficiencies of the prior art by providing numerous safety features for the eyes, ears, and neck of the operator while also increasing the comfort to the operator.

### SUMMARY OF THE INVENTION

In accordance with one aspect, the present disclosure is directed to a welding hood for protecting an operator's face comprising a face protective shield having a front or forward side, which is typically convex in shape, and a back or rearward side, which is typically concave in shape and configured for receiving the operator's face, a viewing opening extending through the shield, wherein the viewing opening is located in a position in front of the eyes of the operator while the hood is fixed to the operator's head. The welding hood also includes an eye box located adjacent the viewing opening. The eye box has at least one wall framing at least a portion of the viewing opening and extends inwardly from the rearward side of the shield. According to one embodiment, a plurality of walls are provided for fully framing or completely surrounding the viewing opening so as to block at least one of reflective arc flashes, sunlight, and shop-lighting from entering the operator's field of view during welding. The welding hood also includes a lens located within the viewing opening for protecting the operator's eyes while allowing the operator to look through the viewing opening and at least one strap for securing the welding hood to the operator's head.

The walls of the eye box can be contoured to fit to the operator's face surrounding the operator's eyes. In particular, the walls can be contoured or can be formed from a flexible material, such as rubber or a foam material, so that the walls fit flush against the operator's face to reduce the amount of sparks, dirt, and/or debris from coming into contact with the operator's eyes.

The lens can comprise a light-filtering lens for filtering light passing through the opening toward the rearward side of the hood. The welding hood can include a holding member for holding the lens, wherein the holding member is capable of pivoting between an open and closed position about the viewing opening. The provision of a pivoting holding member enables the operator to lift the eye lens for better viewing of a fit-up or grind, if needed, or for communication with another person, without having to lift the protective shield from their face. This provides an added safety measure to the welding hood. The holding member can be configured for removably holding the lens so that the lens can be replaced.

According to one embodiment, the at least one strap for securing the welding hood to the operator's head can comprise an elastic strap that wraps around the operator's head. It can be appreciated that the strap can be formed from other known materials. The strap can be adjustable so that the welding hood is firmly secured to the operator's head.

The face protective shield can include a first side portion having a first width configured for extending over at least a portion or completely over the ear of the operator and a second side portion having a second width. According to one embodiment, this second width can be the same as the first width so as to provide added coverage to the operator's ears or side of their face. According to another embodiment, this second width can be less than the first width, wherein the second width is configured to enable air movement about the operator's face. This second width can be designed so as to leave a portion of the operator's ear uncovered so that the operator's hearing is not impaired. It can be appreciated that the welding hood can be molded so that the sides are of equal width, but extended when compared with the prior art, and to include scoring or the like or to be formed from a material that enables one to remove a portion of the side so as to facilitate air flow and uncover a portion of the operator's ear, as desired.

According to one embodiment, the first side portion can comprise a removable attachment portion that can be attached to either a right side or a left side of the face protective shield, depending upon whether the operator is right-handed or left-handed. This flexible attachment portion can be applied to either side of the welding hood, can be added to the already extended side portion, or can even be added to a side portion that has not been extended to provide extra protection. This removable attachment portion enables customization of the welding hood for the operator and provides the operator with additional protection closest to a welding area. According to a further embodiment, the first side portion can be formed from a flexible material. For additional protection, the welding hood can include a flexible attachment portion extending from a bottom portion of the face protective shield to provide protection of the operator's neck. The flexible attachment portions for the side and/or bottom of the shield can be formed from any well-known heat resistant material, such as leather, rubber, heat-resistant plastic, and the like.

In accordance with another aspect, the present disclosure is directed to a welding hood for protecting an operator's face comprising a face protective shield having a concave rearward side for receiving the operator's face, a viewing opening extending through the shield, wherein the viewing opening is located in a position in front of the eyes of the operator while the hood is fixed to the operator's head, a lens located within the viewing opening for protecting the operator's eyes while allowing the operator to look through the viewing opening, and at least one strap for securing the welding hood to the operator's head. The face protective shield can include a first side portion having a first width configured for extending over at least a portion of or completely over the ear of the operator and a second side portion having a second width. According to one embodiment, this second width can be the same as the first width so as to provide added coverage to the operator's ears or side of their face. According to another embodiment, this second width can be less than the first width, wherein the second width is configured to enable air movement about the operator's face. This second width can be designed so as to leave a portion of the operator's ear uncovered so that the operator's hearing is not impaired. It can be appreciated that the welding hood can be molded so that the sides are of equal width, but extended when compared with the prior art, and to include scoring or the like or to be formed from a material that enables one to remove a portion of the side so as to facilitate air flow and uncover a portion of the operator's ear, as desired.

According to one embodiment, the first side portion can comprise a removable attachment portion that can be attached to either a right side or a left side of the face protective shield, depending upon whether the operator is right-handed or left-handed. This flexible attachment portion can be applied to either side of the welding hood, can be added to the already extended side portion, or can even be added to a side portion that has not been extended to provide extra protection. This removable attachment portion enables customization of the welding hood for the operator and provides the operator with additional protection closest to a welding area. According to a further embodiment, the first side portion can be formed from a flexible material. For additional protection, the welding hood can include a flexible attachment portion extending from a bottom portion of the face protective shield to provide protection of the operator's neck. The flexible attachment portions for the side and/or bottom of the shield can be formed from any well-known heat resistant material, such as leather, rubber, heat-resistant plastic, and the like.

The welding hood can further include an eye box located adjacent the viewing opening. The eye box can have at least one wall framing at least a portion of the viewing opening that extends inwardly from the rearward side of the shield. According to one embodiment, the at least one wall can comprise a series of walls that completely frame the viewing opening. The walls can be configured for blocking at least one of reflective arc flashes, sunlight, and shop-lighting from entering the operator's field of view during welding. The walls of the eye box can be contoured or formed from flexible material so that they fit flush about the operator's face to reduce the amount of sparks, dirt, and/or debris from coming into contact with the operator's eyes.

The lens can be located within a holding member capable of pivoting between an open and closed position about the viewing opening. This lens can be removable from the holding member for replacement thereof.

In accordance with yet another aspect, the present disclosure is directed to a welding hood for protecting an operator's face comprising a face protective shield having a rearward side configured for receiving the operator's face and a first side portion having a first width configured for extending over at least a portion of or completely over the ear of the operator and a second side portion having a second width. The welding hood further includes a viewing opening extending through the shield, wherein the viewing opening is located in a position in front of the eyes of the operator while the hood is fixed to the operator's head. An eye box is located adjacent the viewing opening. The eye box has at least one wall, such as a series of walls, framing at least a portion or completely surrounding the viewing opening and extending inwardly from the rearward side of the shield. These walls are configured for blocking at least one of reflective arc flashes, sunlight, and shop-lighting from entering the operator's field of view during welding. A lens is located within the viewing opening for protecting the operator's eyes while allowing the operator to look through the viewing opening. The welding hood also includes at least one strap for securing the welding hood to the operator's head.

According to still another aspect, the present disclosure is directed to a welding hood for protecting an operator's face comprising a face protective shield having a rearward side configured for receiving the operator's face, a viewing opening extending through the shield, and an eye box located adjacent the viewing opening. The eye box has at least one wall framing at least a portion of the viewing opening and extending inwardly from the rearward side of the shield and a lens configured for blocking the viewing opening. The welding hood further comprises a light mounted to the face protective shield wherein the light shines outward from a front side of the face protective shield. The light can be removably located within a light holder and the light holder secured to the face protective shield. According to one embodiment, the light can be removed from the rearward side of the face protective shield for replacement thereof. The light can be an LED or any other known light fixture. It can be appreciated that the light can be battery powered, solar powered, and the like. The eye box comprises a plurality of walls that are configured to fit flush against the operator's face and surround the operator's eyes to reduce the amount of sparks, dirt, and/or debris from coming into contact with the operator's eyes. The lens can be removably located within a holding member capable of pivoting between an open and closed position about the viewing opening and is formed from a light-filtering lens material for filtering light passing through the opening toward the rearward side of the hood. An adjustable strap can be provided for securing the welding hood to an operator's head.

According to one embodiment, a fan can be mounted to the face protective shield. According to another embodiment, a clear protective member can be secured to the eye box at a location adjacent to the viewing opening.

The face protective shield includes a first side portion having a first width configured for extending over at least a portion of an ear of the operator and a second side portion having a second width. According to one design, the second width of the second side portion can be substantially equal to the first width of the first side portion so that both the first side portion and the second side portion extend over the ears of the operator. According to another design, the second width of the second side portion can less than the first width of the first side portion, wherein the second width is configured to enable air movement about the operator's face.

According to another aspect, the present disclosure is directed to a welding hood for protecting an operator's face comprising a face protective shield having a rearward side configured for receiving the operator's face, a first side portion having a first width configured for extending over at least a portion of an ear of the operator and a second side portion having a second width, and a viewing opening extending through the shield. The viewing opening can be located in a position in front of the eyes of the operator while the hood is fixed to the operator's head. A lens can be located within the viewing opening for protecting the operator's eyes while allowing the operator to look through the viewing opening. A fan is mounted to the face protective shield for supplying air to the operator's face and at least one strap is provided for securing the welding hood to the operator's head. The fan can be mounted to the rearward side of the face protective shield. According to one embodiment, a light can be mounted to the face protective shield. The welding hood further includes an eye box located adjacent the viewing opening. The eye box has at least one wall framing at least a portion of the viewing opening and extending inwardly from the rearward side of the shield. The at least one wall is configured for blocking at least one of reflective arc flashes, sunlight, and shop-lighting from entering the operator's field of view during welding. According to one embodiment, the eye box can include a clear protective member mounted therein configured to provide eye protection to the operator. According to one design, the second width of the second side portion can be less than the first width of the first side portion, wherein the second width is configured to enable air movement about the operator's face. The widths of the side portions can be customized in accordance with the dominant hand of the operator.

According to still another aspect, the present disclosure is directed to a welding hood for protecting an operator's face comprising a face protective shield having a rearward side configured for receiving the operator's face, a viewing opening extending through the shield, said viewing opening located in a position in front of the eyes of the operator while the hood is fixed to the operator's head, and an eye box located adjacent the viewing opening, said eye box having at least one wall framing at least a portion of the viewing opening and extending inwardly from the rearward side of the shield, wherein the walls are configured for blocking at least one of reflective arc flashes, sunlight, and shop-lighting from entering the operator's field of view during welding. The welding hood further includes a protective member secured to the eye box and located adjacent the viewing opening at a position in front of the eyes of the operator while the hood is fixed to the operator's eyes and a holding member pivotally secured to a front side of the face protective shield. The holding member configured for holding a lens at a location adjacent to the viewing opening. A fan can be secured to the rearward side of the face protective shield, a light mounted to the face protective shield wherein the light shines outward from a front side of the face protective shield, and at least one strap for securing the welding hood to the operator's head. According to one embodiment, the face protective shield can include a first side portion having a first width configured for extending over at least a portion of the ear of the operator and a second side portion having a second width that is less than the first width of the first side portion, wherein the second width is configured to enable air movement about the operator's face.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a front perspective view of a welding hood in accordance with the prior art.

FIG. 2 is a back perspective view of the welding hood of FIG. 1, in accordance with the prior art.

FIG. 3 is a side view of the welding hood of FIG. 1, positioned on the head of an operator in accordance with the prior art.

FIG. 4 is a front perspective view of the welding hood in accordance with an embodiment of the present invention.

FIG. 5 is a back perspective view of the welding hood of FIG. 4 in accordance with an embodiment of the present invention.

FIG. 6 is a top view of the welding hood of FIG. 4 in accordance with an embodiment of the present invention.

FIG. 7 is a bottom view of the welding hood of FIG. 4 in accordance with an embodiment of the present invention.

FIG. 8 is a left view of the welding hood of FIG. 4 in accordance with an embodiment of the present invention.

FIG. 9 is a right view of the welding hood of FIG. 4 in accordance with an embodiment of the present invention.

FIG. 10 is a front view of the welding hood of FIG. 4 in accordance with an embodiment of the present invention.

FIG. 11 is a back view of the welding hood of FIG. 4 in accordance with an embodiment of the present invention.

FIG. 12 is a front perspective view of a welding hood in accordance with another embodiment of the present invention.

FIG. 13 is a back perspective view of the welding hood of FIG. 12 in accordance with an embodiment of the present invention.

FIG. 14 is a top view of the welding hood of FIG. 12 in accordance with an embodiment of the present invention.

FIG. 15 is a bottom view of the welding hood of FIG. 12 in accordance with an embodiment of the present invention.

FIG. 16 is a left view of the welding hood of FIG. 12 in accordance with an embodiment of the present invention.

FIG. 17 is a right view of the welding hood of FIG. 12 in accordance with an embodiment of the present invention.

FIG. 18 is a front view of a welding hood in accordance with an embodiment of the present invention.

FIG. 19 is a side view of a welding hood in accordance with yet another embodiment of the present invention.

FIG. 20A is a front perspective view of the welding hood in accordance with another embodiment of the present invention.

FIG. 20B is a front perspective view of the welding hood of FIG. 20A, wherein the holding member including the lens has been flipped to the open position in accordance with an embodiment of the invention.

FIG. 21A is a back perspective view of the welding hood of FIG. 20A in accordance with an embodiment of the invention.

FIG. 21B is a back perspective view taken from a bottom angle of the welding hood of

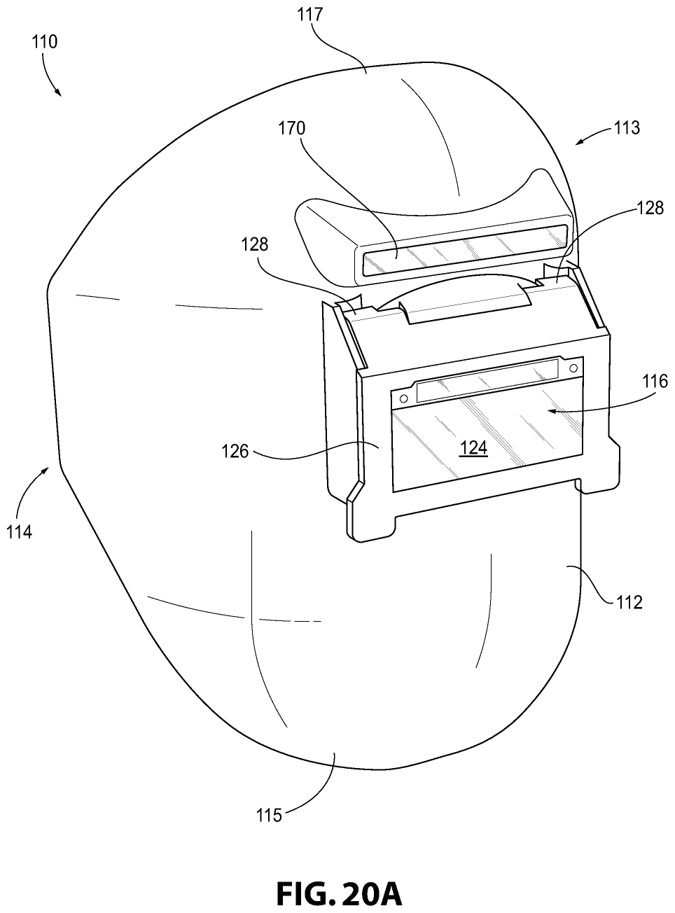

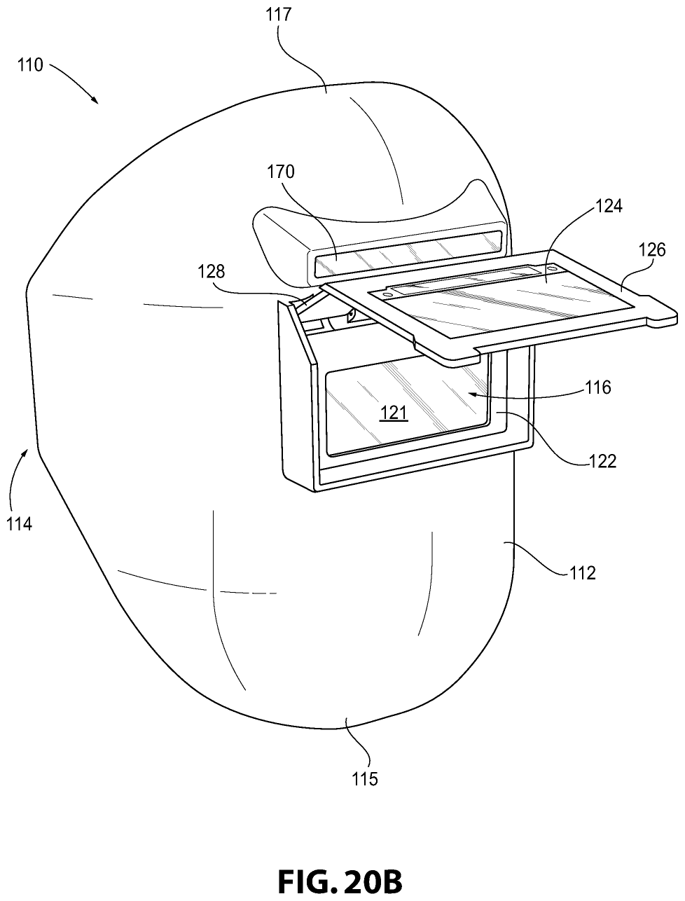

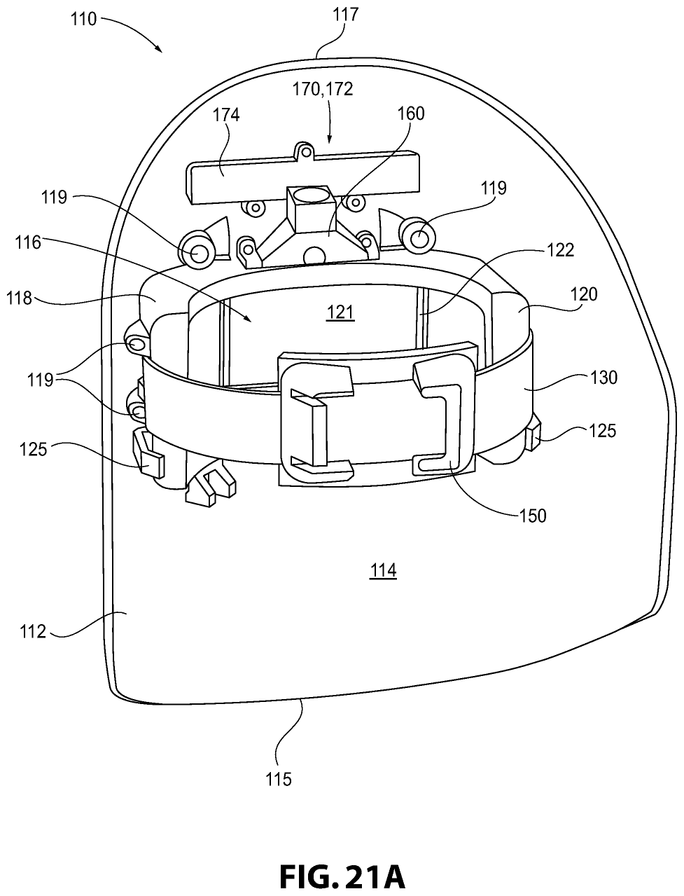

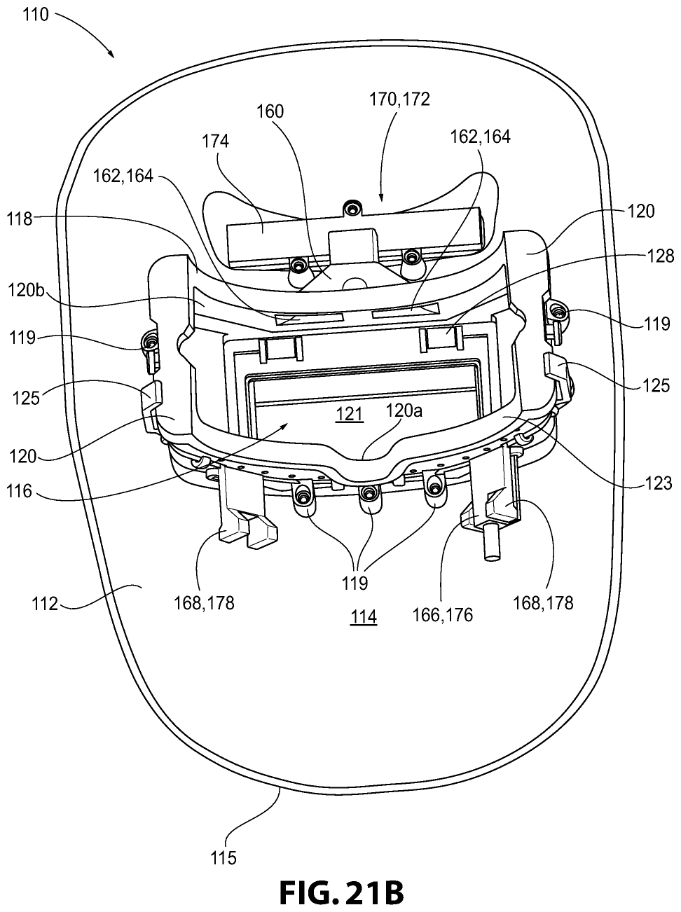

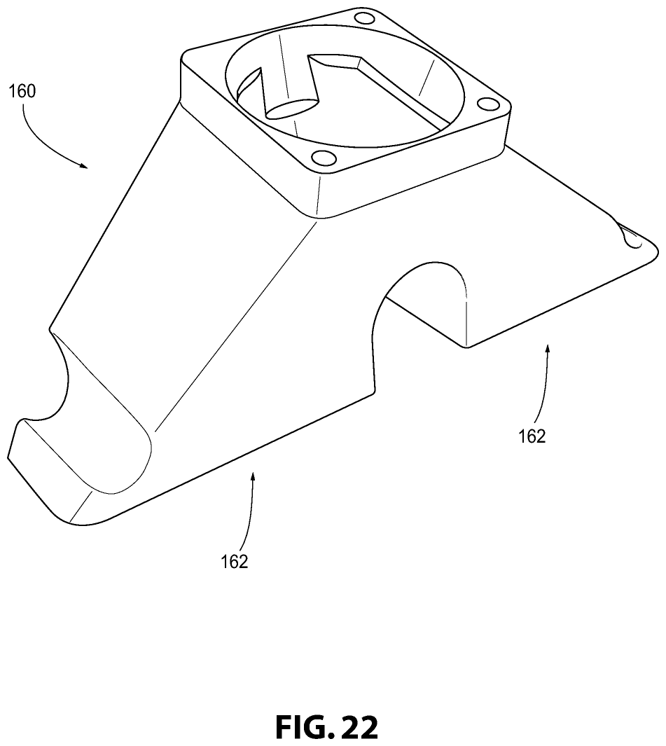

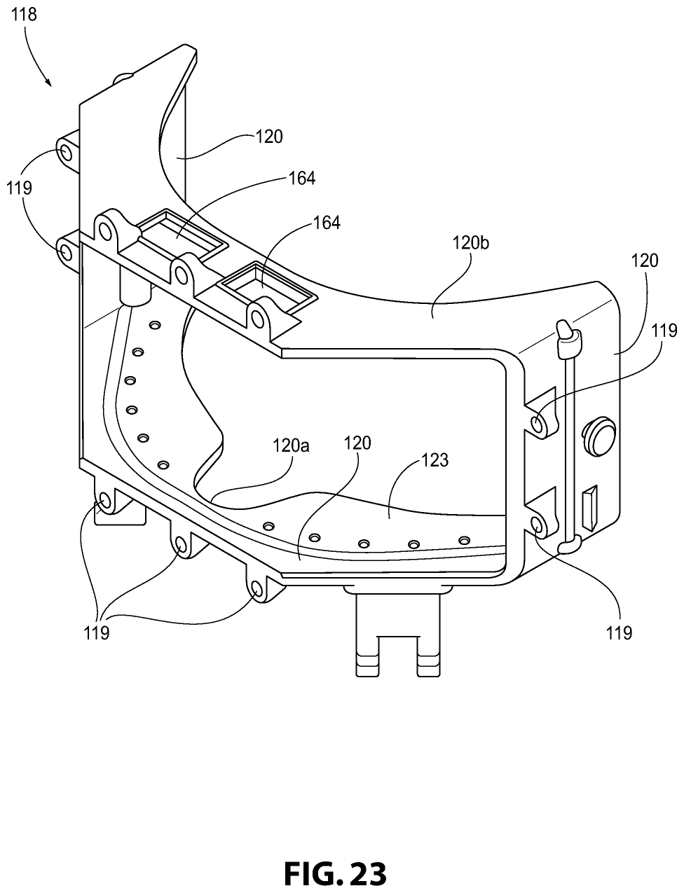

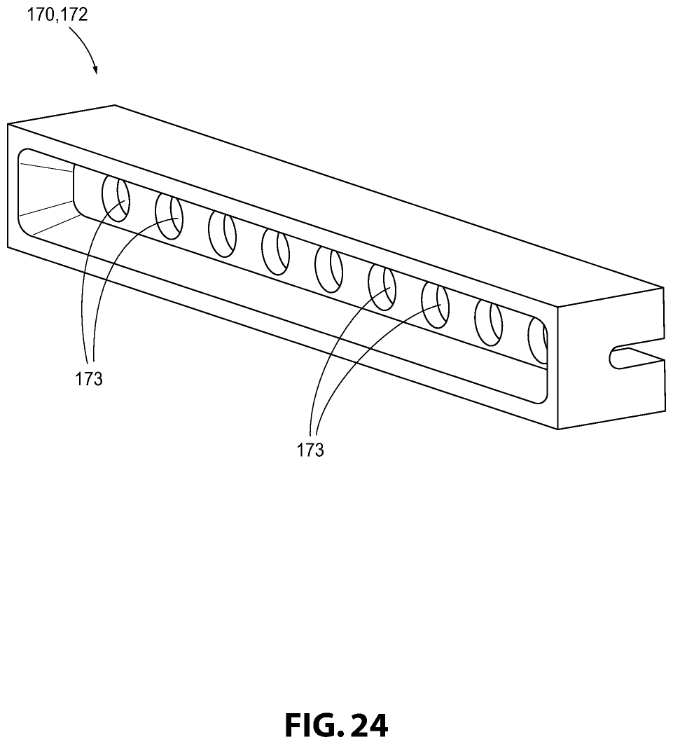

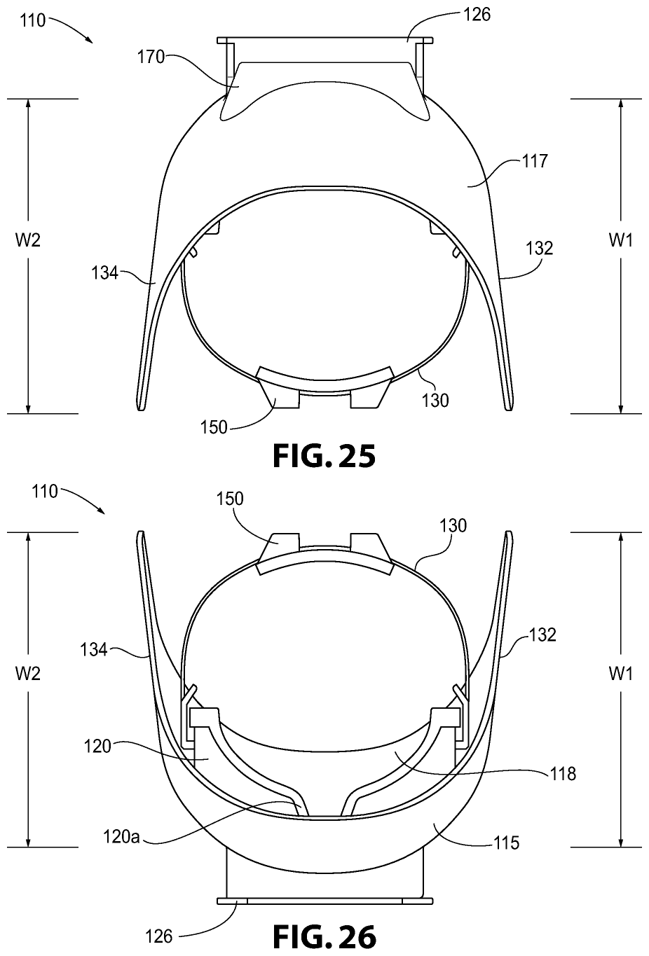

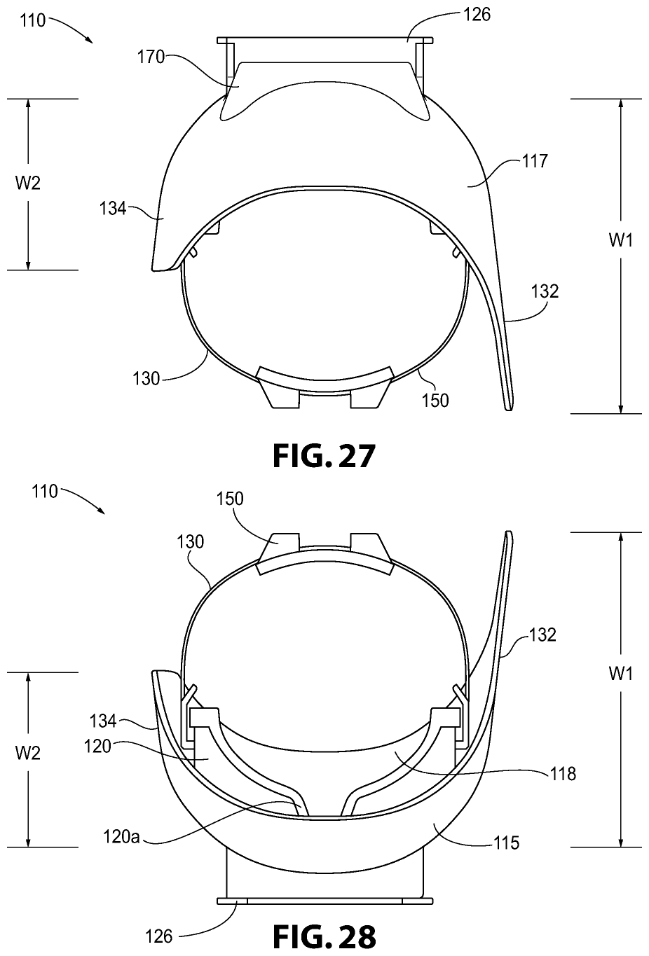

FIGS. 20A and 21A with the holding strap removed in accordance with an embodiment of the invention.

FIG. 22 is a perspective view of a fan duct used in the welding hood shown in FIGS. 21A and 21B in accordance with an embodiment of the invention.

FIG. 23 is a perspective view of the eye box of the welding hood shown in FIGS. 21A and 21B in accordance with an embodiment of the invention.

FIG. 24 is a perspective view of the light holder of the welding hood of FIGS. 20A-21B in accordance with an embodiment of the invention.

FIG. 25 is a top view of the welding hood of FIG. 20A in accordance with an embodiment of the invention.

FIG. 26 is a bottom view of the welding hood of FIG. 20A in accordance with an embodiment of the invention.

FIG. 27 is a top view of the welding hood of FIG. 20A in accordance with another embodiment of the invention.

FIG. 28 is a bottom view of the welding hood of FIGS. 20A and FIG. 27 in accordance with an embodiment of the invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

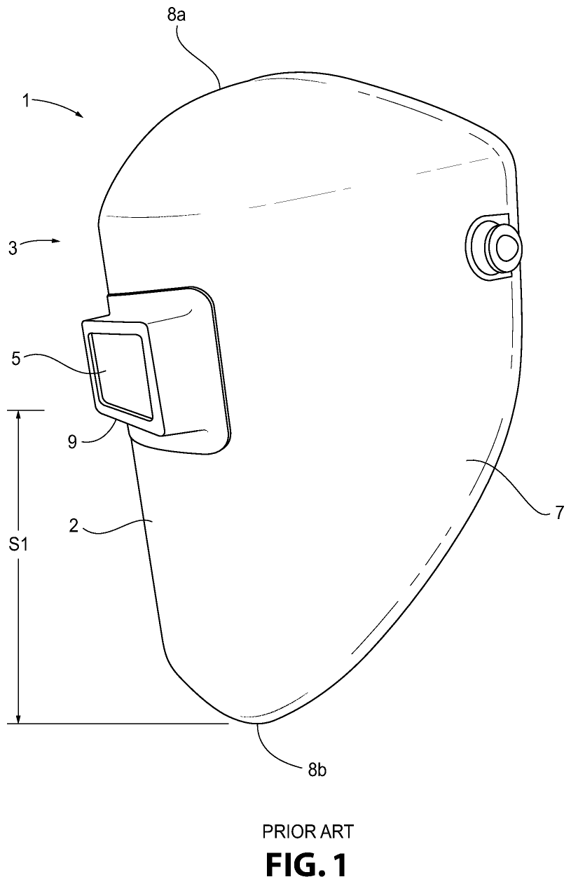

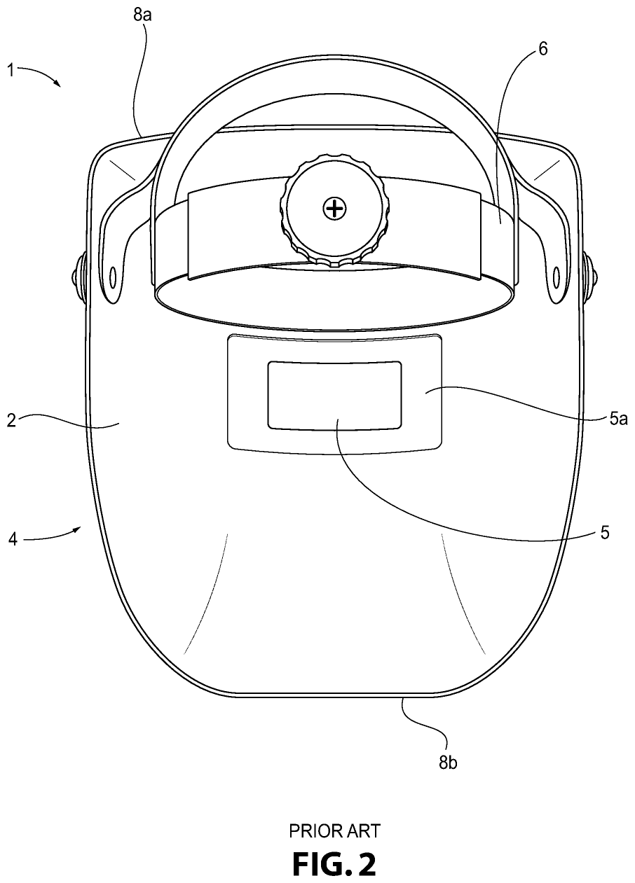

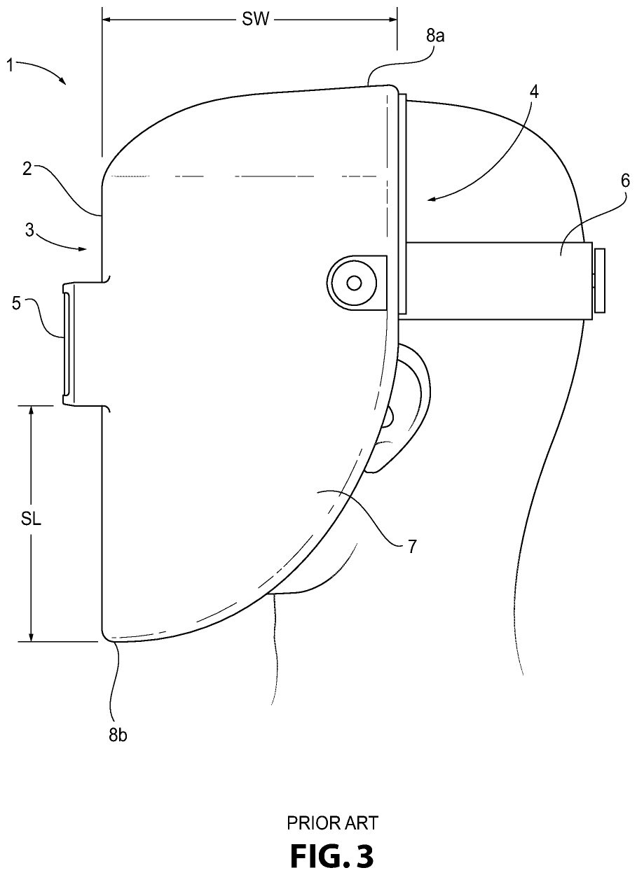

Reference is now made to FIGS. 1-3, which show one type of welding hood, generally indicated as 1, in accordance with the prior art. The welding hood 1 comprises a face protective shield 2, having a forward or front side 3, that is convex in shape, and a rearward or back side 4 that is concave in shape and configured for receiving an operator's face, a top 8a and a bottom 8b. A viewing opening or window 5 is provided so as to enable the operator to see what is being welded. A strap 6 is provided for holding the hood about the operator's face. As can be seen from FIG. 2, the area or frame 5a surrounding the viewing opening 5 is either flush with or extends slightly inward with respect to the rearward side 4 of the shield 2. As shown in FIG. 3, the welding hood 1 includes side portions 7 that only partially cover the operator's ears. Also, as can be seen in FIGS. 1-3 the bottom 8b of the hood 1 or face shield 2, does not extend very far down, leaving a significant portion of the operator's neck exposed.

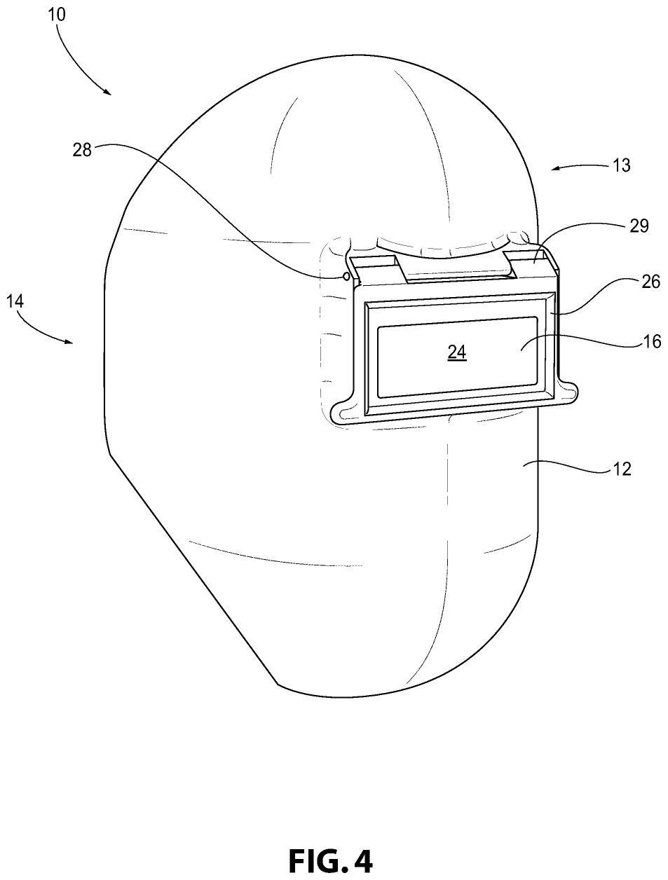

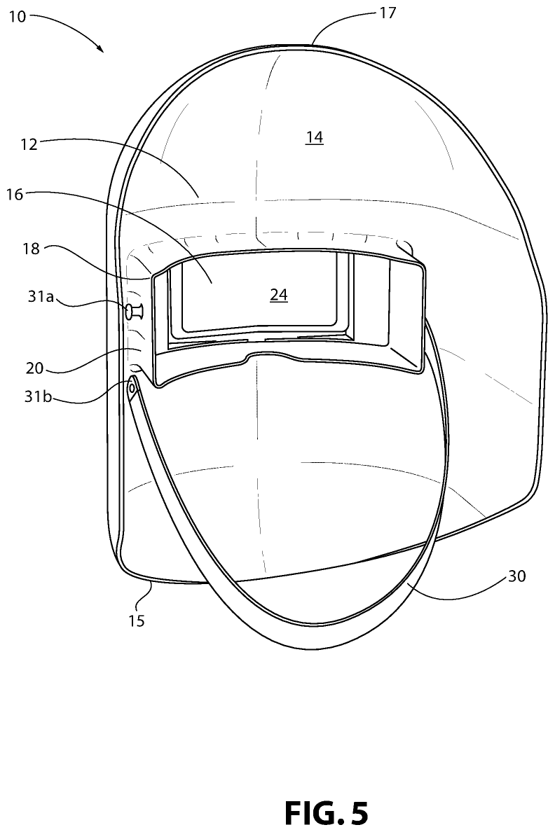

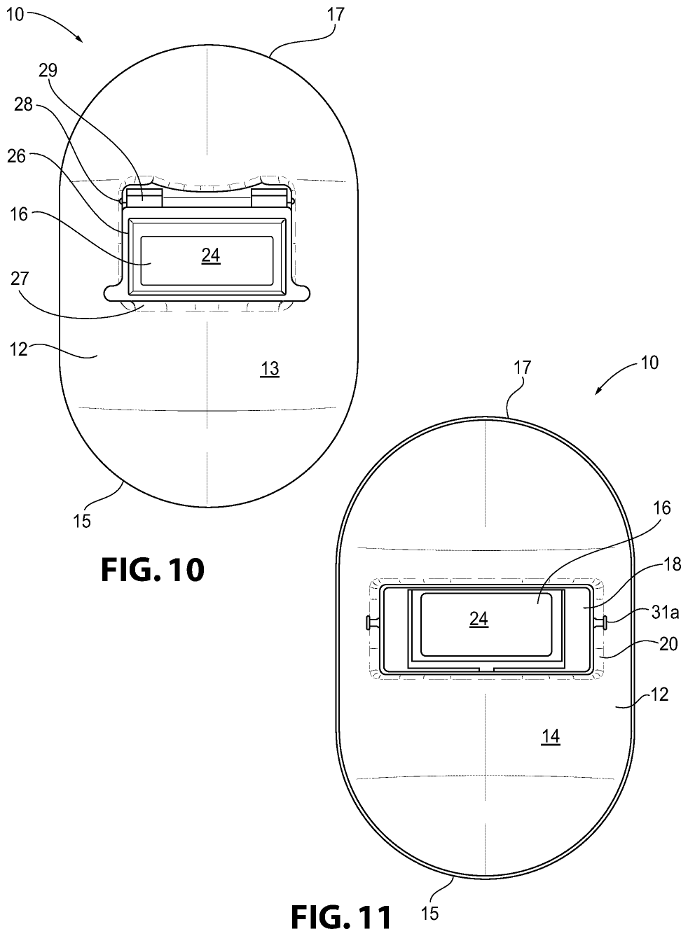

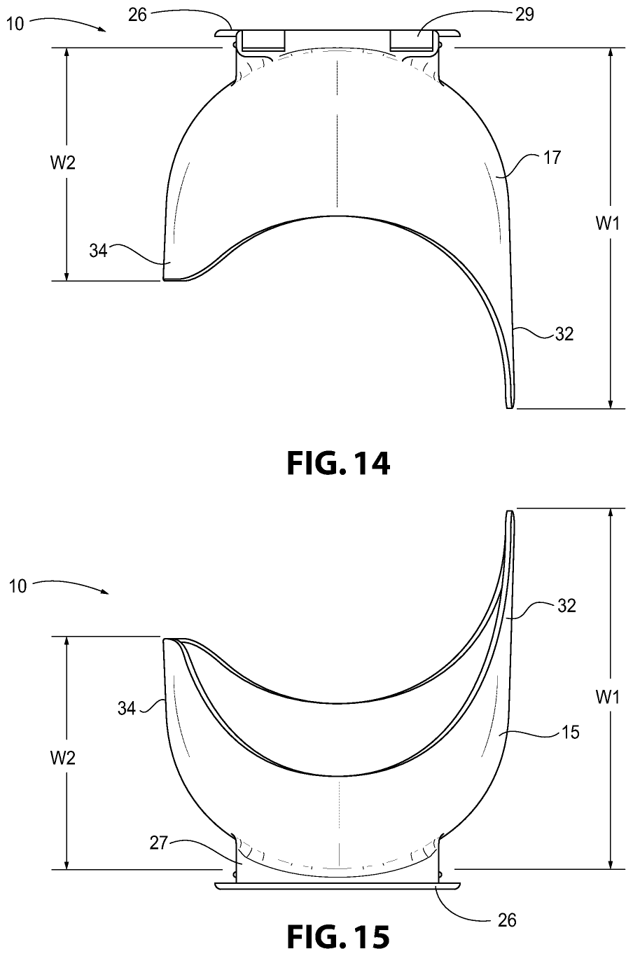

Reference is now made to FIGS. 4-19, which are directed to the welding hood, generally indicated as 10, for protecting an operator's face, as well as the operator's ears and neck, in accordance with the present disclosure. The welding hood 10 comprises a face protective shield 12 having a forward or front side 13, which is typically convex in shape, a rearward or back side 14, which is typically concave in shape, and configured for receiving the operator's face, a bottom portion 15, and a top portion 17. A viewing opening 16 extends through the face shield 12. The viewing opening 16 is located in a position in front of the eyes of the operator when the hood 10 is fixed to the operator's head. It can be appreciated that the opening 16 can have any shape, including, but not limited to, a square, rectangle, oval, a pair of circles, and the like. The welding hood 10 also includes an eye box 18, extending from the rearward or back side 14 of the hood 10, as shown in FIGS. 5, 11, and 13. The eye box 18 is located adjacent the viewing opening 16. It can also be appreciated that the eye box 18 can have any shape, including, but not limited to, a square, rectangle, oval, a pair of circles, and the like.

With continuing reference to FIGS. 5, 11, and 13, the eye box 18 has at least one wall 20 framing at least a portion of the viewing opening 16. The at least one wall 20 extends inwardly from the rearward side 14 of the shield 12. According to one embodiment, a plurality of walls 20 are provided for fully framing or completely surrounding the viewing opening 16 so as to block at least one of reflective arc flashes, sunlight, shop-lighting, and the like, from entering the operator's field of view during welding. The welding hood 10 also includes a lens 24 located within the viewing opening 16 for protecting the operator's eyes while allowing the operator to look through the viewing opening 16.

Still referring to FIGS. 5, 11, and 13, the walls 20 of the eye box 18 can be contoured to fit to the operator's face and surround the operator's eyes. In particular, the walls 20 can be contoured or can be formed from a flexible material, such as rubber or a foam material, so that the walls 20 fit flush against the operator's face to reduce the amount of sparks, dirt, debris and the like, from coming into contact with the operator's eyes. It can be appreciated that the walls 20 can be formed from an inflexible or rigid material, such as wood, plastic, or stiff rubber material, wherein the rigid material is shaped or contoured to fit an operator's face. It can also be appreciated that the walls 20 can be formed from a partially flexible material, such as a rubber material or a heat resistant foam rubber material, that is sufficiently flexible to conform to the operator's face, but rigid enough to provide blockage against arc flashes, light, sparks, dirt, debris, and the like.

The lens 24 can comprise a light-filtering lens for filtering light passing through the opening toward the rearward side 14 of the hood 10. The welding hood 10 can include a holding member 26 for removably holding the lens 24. The holding member 26 includes a pivoting portion 29 that is capable of pivoting via one or more pivot screws 28, between an open and closed position about the viewing opening 16. The provision of a pivoting holding member 26 enables the operator to lift the holding member 26 and lens 24 for better viewing of a fit-up or grind, if needed, or for communication with another person, without having to lift the protective shield 12 from their face. This provides an added safety measure to the welding hood 10. The holding member 26 can be configured for removably holding the lens 24 so that the lens can readily be removed and replaced with another light-filtering lens, whether for replacement due to damage or for replacement with a darker lens or with a clear lens.

As shown in FIGS. 5 and 13, at least one strap 30 is provided for securing the welding hood 10 to the operator's head. The at least one strap 30 can comprise an elastic strap that wraps around the operator's head. According to one embodiment, a pair of extending members 31a, such as bolts, screws, and the like, can be provided on opposing walls 20 of the eye box 18 and the strap 30 can be secured to the extending members 31a, such as with hooks 31b located at the ends of the strap 30. This arrangement enables easy attachment and removal of the strap 30 about the operator's head. It can be appreciated that the strap 30 can be secured to the eye box by other well-known means, such as rivets, screws, and the like. The strap 30 can also be secured to the face shield 12, itself, by known attachment arrangements. The elastic strap 30 can be adjustable, such as with a double band and a movable buckle or any other well-known means to adjust the strap, so as to securely hold the hood 10 in place for both safety and comfort. According to one embodiment, the strap 30 can be formed from an elastic material, similar in size and shape to a strap used in ski goggles or scuba masks. The strap 30 is designed to hold the hood 10 in place, regardless of the position and/or movement of the operator's head. It can be appreciated that the strap can be formed from other known materials and can have other known designs.

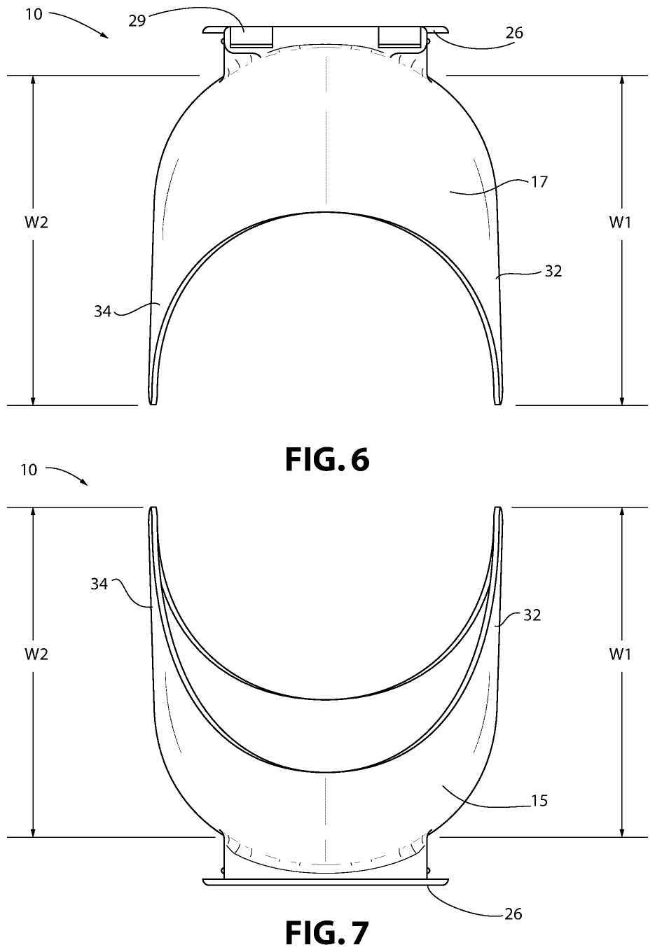

Referring now to FIGS. 6-9 and 14-17, the face protective shield 12 can include a first side portion 32 having a first width “W1” configured for extending over at least a portion of or completely covering the ear of the operator and a second side portion 34 having a second width “W2”. According to the embodiment shown in FIGS. 3-11, the first width W1 can be substantially equal to the second width “W2”. This particular design provides added coverage to the operator's ears and/or sides of their face, especially when compared with the prior art design, as illustrated in FIGS. 1-3. Prior art welding hood designs, such as shown in FIGS. 1-3, typically have a standard width “SW” from the front of the lens holder back to the rear edge on the side of about 7-7⅛ inches. As can be seen in FIG. 3, this design leaves a substantial portion of the operator's ear and/or face exposed. The welding hood of the present invention has at least one or both sides extended to a width “W1” and “W2” of approximately 8-9 inches, or 8¾ inches, from the front of the lens holder 26 back to the rear edge on the sides 32 and/or 34 so as to provide extra protection to the operator.

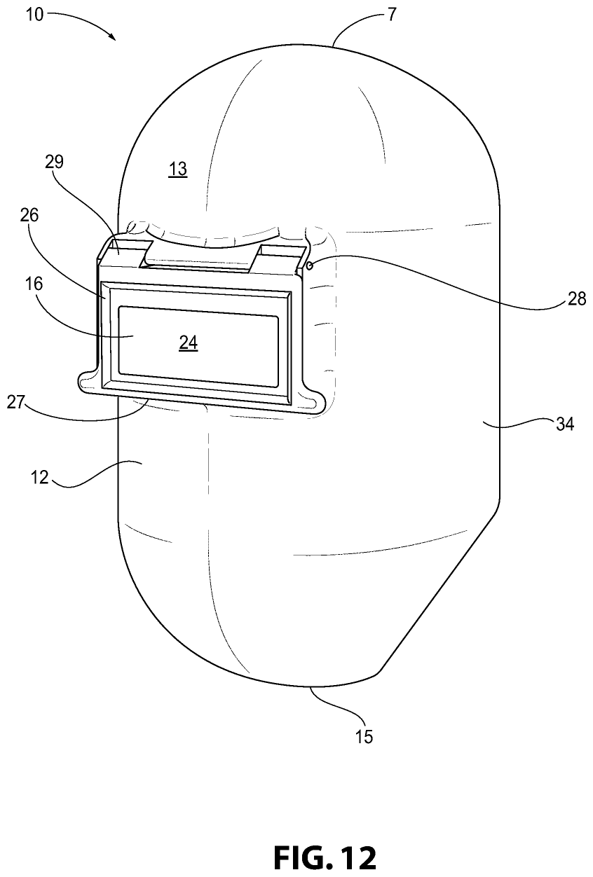

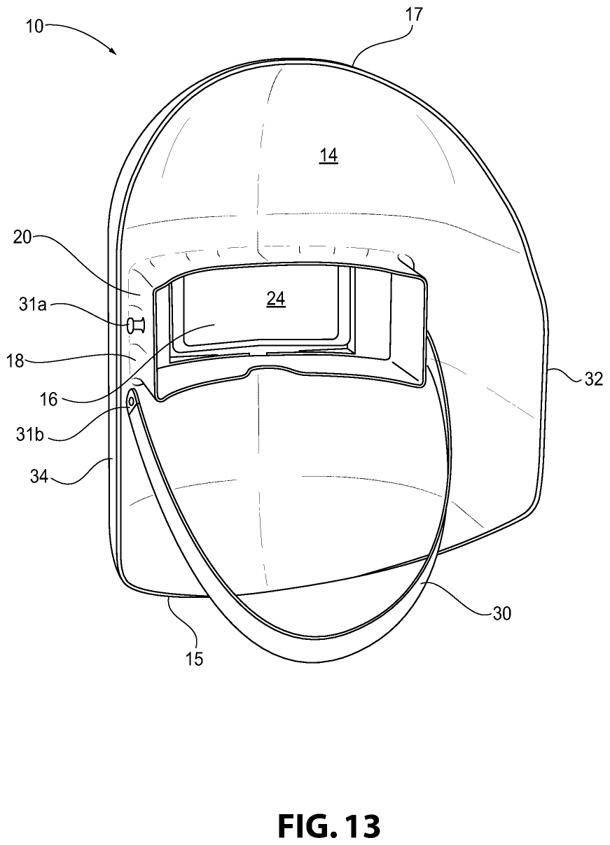

According to the embodiment shown in FIGS. 12-17, the second width “W2” can be less than the first width “W1”, wherein the second width “W2” is configured to enable air movement about the operator's face. This second width “W2” can also be designed so as to leave a portion of the operator's ear uncovered so that the operator's hearing is not impaired.

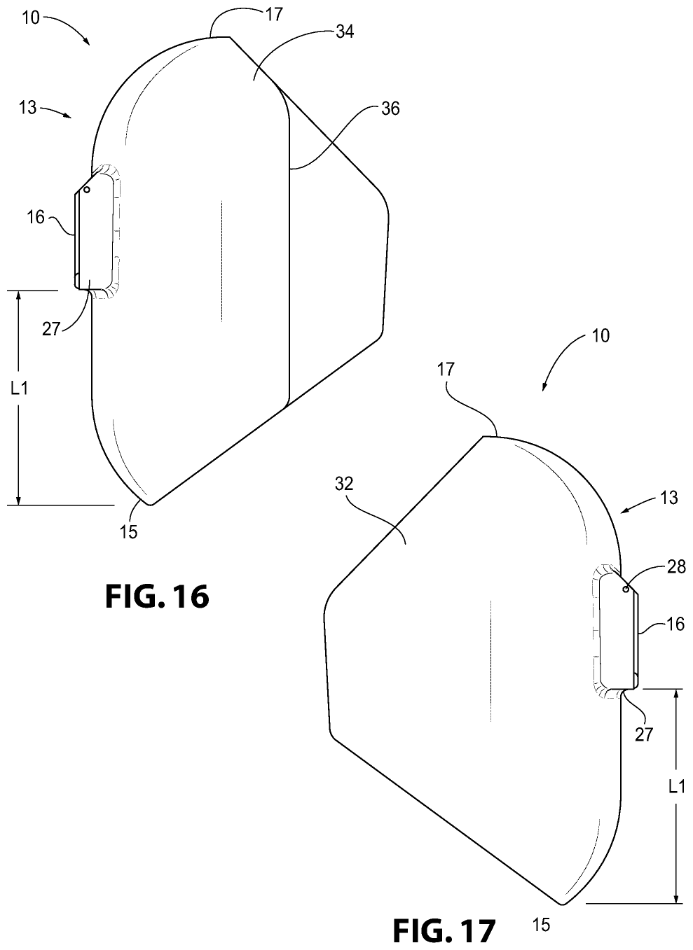

It can be appreciated that the welding hood 12 can be molded so that the first and second widths “W1” and “W2” of the first side 32 and second side 34 are of equal width, but extended when compared with the prior art. The first and second sides 32, 34 can include scoring 36, as shown in FIG. 16, or the like, to facilitate removal of a portion of one or both sides 32, 34, as needed. Alternatively, the welding hood can be formed from a material that enables one to remove a portion of one or both sides 32, 34 so as to facilitate air flow under the welding hood 10 and uncover a portion of the operator's ear and/or face, as desired.

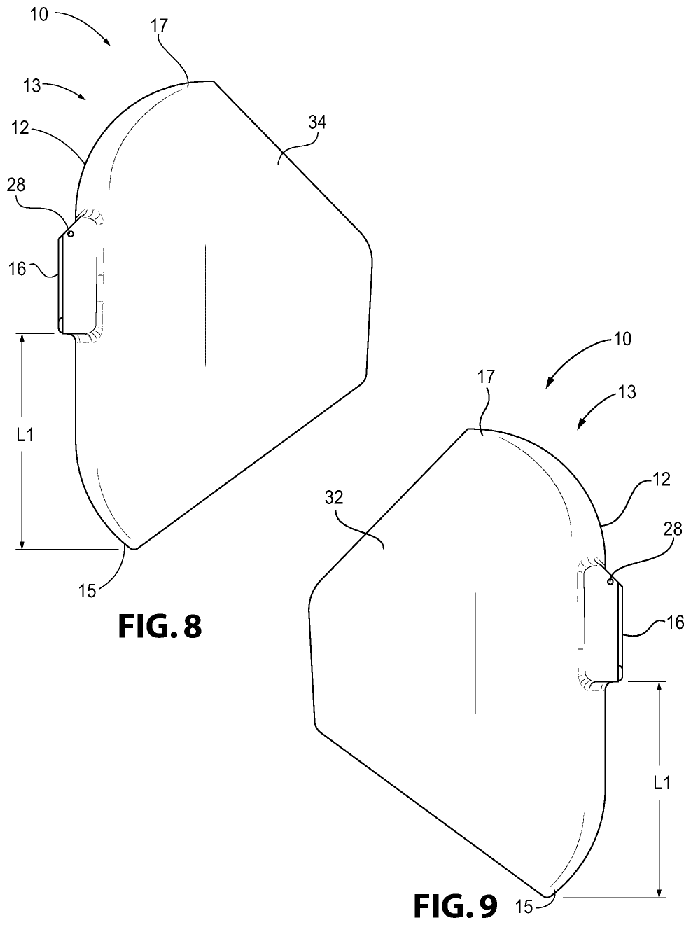

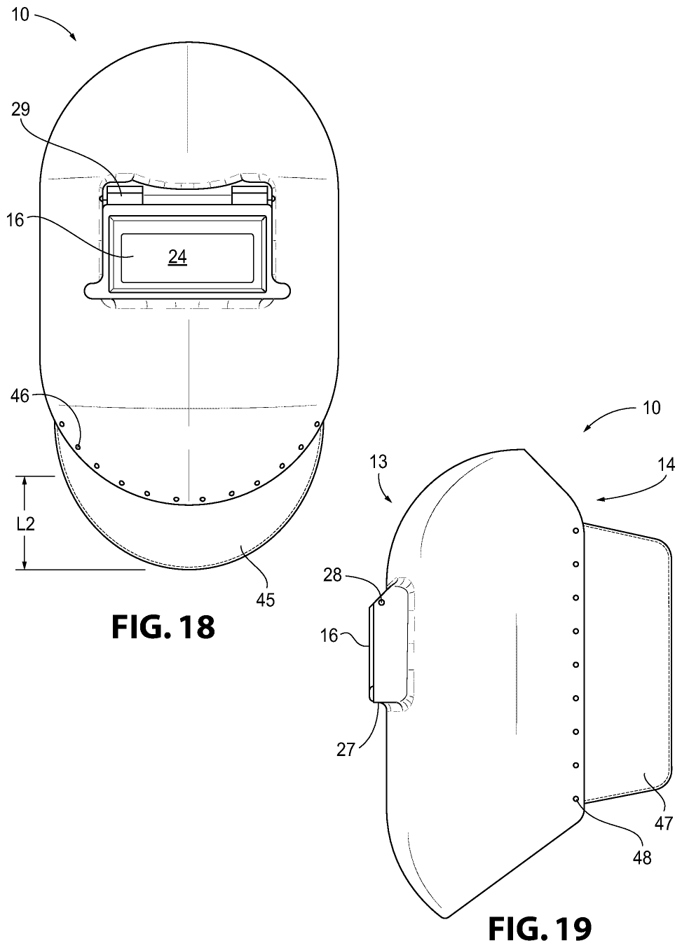

Referring back to FIGS. 1-3, the prior art hood 1 has a bottom portion 8b that extends from the bottom 9 of the viewing opening 5 for a length “S1” of approximately 7½ inches but curls under the chin, leaving a significant portion of the neck exposed, as shown in FIG. 3. In accordance with one embodiment of the present invention and with reference to FIGS. 8, 9, 16, and 17, the welding hood 10 can be designed to have a length “L1” of rigid material of approximately 6 to 7 inches or approximately 6½ inches, from the bottom 27 of the lens holding member 26, and, as shown in FIG. 18, a bottom attachment portion 45, such as leather or similar material, can be attached to the bottom portion 15 of the face shield 12 to provide additional protection of the operator's neck. This bottom attachment portion 45 can have a length “L2” of approximately 2-3 inches, or 2½ inches, and can be attached to the bottom portion 15 of the face shield 12 by any well-known means 46, such as snaps/rivets, hook and loop, auto tape, as the like, such that this bottom attachment portion drapes and/or flexes for further protection of the neck without curling under the chin. It can be appreciated that the flexible attachment portion 45 for the bottom of the face shield 12 can be formed from any well-known heat resistant material, such as leather, rubber, heat-resistant plastic, and the like. According to one example, the flexible bottom attachment portion 45 can be a three-by-twelve-inch suede split or similar material attached to the bottom portion 15 of the hood.

With reference to FIG. 19, according to one embodiment, the first or second side portion 32, 34 can comprise a removable side attachment portion that can be attached to either a right side or a left side of the face protective shield 12, depending upon whether the operator is right-handed or left-handed. This removable attachment portion 47 can be applied to either side 32, 34 of the face protective shield 12 of the welding hood 12 or it can be applied to both sides 32, 34, if desired. It can be appreciated that the removable attachment portion 47 can be added to the already extended side portion 32, 34 to provide extra protection, or can be added to a side portion that has not been extended to provide protection as needed. This removable attachment portion 47 enables customization of the welding hood 10 for the operator and provides the operator with additional protection closest to a welding area. The removable attachment portion 47 can be formed from a flexible material, such as any well-known heat resistant material, including but not limited to leather, rubber, suede, heat-resistant plastic, and the like and can be secured by any well-known securing means 48, such as snaps, hook and loop, and the like. The securing means 48 can be provided on both sides of the face protective shield 12 so that the attachment portion 47 can be secured to either side 32, 34 of the face shield 12, enabling customization of the welding hood 10, depending upon whether the operator is a right-handed or left-handed welder and/or to provide the operator with additional protection closest to a welding area.

Reference is now made to FIGS. 20A, 20B, 21A, 21B and 25-28 which are directed to another embodiment of a welding hood, generally indicated as 110, for protecting an operator's face, as well as the operator's ears and neck, in accordance with the present disclosure. The welding hood 110 comprises a face protective shield 112 having a forward or front side 113, which is typically convex in shape, a rearward or back side 114, which is typically concave in shape, and configured for receiving the operator's face, a bottom portion 115, and a top portion 117. A viewing opening 116 extends through the face shield 112. The viewing opening 116 is located in a position in front of the eyes of the operator when the hood 110 is fixed to the operator's head. It can be appreciated that the opening 116 can have any shape, including, but not limited to, a square, rectangle, oval, a pair of circles, and the like. The welding hood 110 also includes an eye box 118, extending from the rearward or back side 114 of the hood 110, as shown in FIGS. 21A, 21B, and 23. The welding hood 110 is molded to accept the configuration of the eye box 118. The eye box 118 is located adjacent the viewing opening 116. It can be appreciated that the eye box 118 can have any shape, including, but not limited to, a square, rectangle, oval, a pair of circles, and the like. It can also be appreciated that the eye box 118 can be integrally formed with the face shield 112 during a molding process. Alternatively, the eye box 118 can be separately attached by well-known attachment members, such as bolts or screws, cooperating with openings or access areas 119 spaced at various location along the edge of the eye box 118. According to one design, the wall 120 of the eye box 118 is fitted with a groove to slip on the eye box 118 and is secured with a clip 125 on each side for ease of changing or adjusting the gasket 123.

With reference to FIG. 21B and FIG. 22, and similar to the design shown in FIGS. 5, 11, and 13, as discussed above, the eye box 118 has at least one wall 120 framing at least a portion of the viewing opening 116. The at least one wall 120 extends inwardly from the rearward side 114 of the shield 112. According to one embodiment, a plurality of walls 120 are provided for fully framing or completely surrounding the viewing opening 116 so as to block at least one of reflective arc flashes, sunlight, shop-lighting, and the like, from entering the operator's field of view during welding. The welding hood 110 also includes a lens 124 located within the viewing opening 116 for protecting the operator's eyes while allowing the operator to look through the viewing opening 116.

With reference to FIG. 20B and with further reference to FIGS. 21B and 22, a clear protective member 121, such as formed from plastic, glass, and the like, can also be provided within a frame 122 of the eye box 118. This protective member 121 will provide additional protection to the eyes of the operator and/or eliminates the need for an operator to wear protective goggles or glasses.

As discussed in detail above in relation to FIGS. 5, 11, and 13, and with further reference to FIG. 22, the walls 120 of the eye box 118 can be contoured to fit to the operator's face and surround the operator's eyes. The walls 120 can include a nose portion 120a configured to fit about an operator's nose. The walls 120 can be formed from a flexible material, such as rubber or a foam material, so that the walls 120 fit flush against the operator's face to reduce the amount of sparks, dirt, debris and the like, from coming into contact with the operator's eyes. A separate gasket 123 can be removably secured to the walls of the eye box 118, which would enable replacement of the gasket 123 due to wear.

The lens 124 can comprise a light-filtering lens for filtering light passing through the opening toward the rearward side 114 of the hood 110. The welding hood 110 can include a holding member 126 for removably holding the lens 124. The holding member 126 includes a pivoting arrangement 128 that is capable of pivoting, between an open and closed position about the viewing opening 116. The provision of a pivoting holding member 126 enables the operator to lift the holding member 126 and lens 124 for better viewing of a fit-up or grind, if needed, or for communication with another person, without having to lift the protective shield 112 from their face. Upon lifting of the holding member 126, the operator's eye still remain protected by the protective member 121. These features provide added safety measures to the welding hood 110. The holding member 126 can be configured for removably holding the lens 124 so that the lens can readily be removed and replaced with another light-filtering lens 124, whether for replacement due to damage or for replacement with a darker lens or with a clear lens.

At least one strap 130, shown in FIG. 21A, is provided for securing the welding hood 10 to the operator's head. The at least one strap 130 can comprise an elastic strap that wraps around the operator's head. According to one embodiment, the strap 130 can be secured to opposing walls 120 of the eye box 118 using any well-known securing members. An adjustable buckle 150 or the like, can be provided for adjusting the fit of the hood 110 about the user's head. The strap 130 is designed to hold the hood 110 in place, regardless of the position and/or movement of the operator's head. It can be appreciated that the strap can be formed from other known materials and can have other known designs.

Reference is now made to FIGS. 21B and 23, which show a battery operated fan 160. The fan 160 can be secured to the rearward side 114 of the face shield 112. According to one embodiment, the fan 160 is secured above the eye box 118 so that air outlet openings 162 of the fan extend through openings 164 of a top wall 120b of the eye box 118 so that cooling air is be directed downward onto the operator's face. According to one embodiment, the battery (not shown) for the fan 160 can be located within an opening 152 of the adjustable buckle 150. Positioning the battery within the buckle 150 and on the strap 130 has the advantage in that is reduces the weight on the welding hood 110, itself. One example for the battery can be a 30C lithium battery, however, it can be appreciated that other types of batteries can be used for powering the fan 160. Wires (not shown) can be provided to attach the battery to the fan 160 and to attach a switch 166 provided in switch holder 168 to cause the fan to turn on and off. It can be appreciated that other power sources, such as solar power, and the like can be used to supply energy to the fan 160. It can also be appreciated that various types of switches and various sizes of fans can be used in the welding hood 110. It can further be appreciated that different designs and/or methods can be used for attaching the battery or solar device to the fan 160 and for attaching the switch 166 to the fan 160, either by attaching the switch 166 directly to the welding hood 110 and/or by providing the switch 166 at a location that is remote from the welding hood 110.

Referring back to FIGS. 20A, 21A, and 21B, and with further reference to FIG. 24, a light 170, such as a battery or solar powered light, such as an LED light, can be provided within a light holder 172, as shown in FIG. 24. The light is configured so that it shines through openings 173 in the light holder 172 and through the front or forward side 114 of the face shield 112. A backing plate 174, shown in FIG. 21B, can be removably secured to the back of the light holder 172 to secure the bulb or bulbs within the light holder 172. The backing plate 174 can be accessed from the back or rearward side 116 of the face shield 112. The bulb or bulbs can be changed, if needed, by the removal of backing plate 174. Typically, when extra light is needed during a welding operation, a second operator would shine a light on the workpiece to assist the welder. The provision of the light 170 on the welding hood itself 110 eliminates the need for a second operator to hold a light and eliminates the danger that this second operator is injured by sparks, dirt, or debris during the welding operation. According to one embodiment, the battery used for supplying power to the fan 160 can also be used to supply power to the light 170. Alternatively, a separate battery or power source can be used to supply power to the light 170. Wires (not shown) can be provided to attach the battery to the light 170 and to attach a switch 176 provided in switch holder 178 to cause the light to turn on and off. It can be appreciated that other power sources, such as solar power, and the like, can be used to supply energy to the light 170. It can also be appreciated that various types of switches 176 and various types/sizes of lights 170 can be used in the welding hood 110. It can further be appreciated that different designs and/or methods can be used for attaching the battery or solar power source to the light 170 and for attaching the switch 176 directly to the welding hood 110 and/or by providing the switch 176 at a location that is remote from the welding hood 110. Also, a separate fan switch 166 and a separate light switch 176 can be located in separate switch holders 168, 178, respectively, to separately operate the fan 160 and the light 170. Alternatively, a single switch, located in one of the switch holders, can be provided to operate both the fan 160 and the light 170.

Referring now to FIGS. 25-28, similar to the designs shown in FIGS. 6-9 and FIGS. 14-17, the face protective shield 112 can include a first side portion 132 having a first width “W1” configured for extending over at least a portion of or completely covering the ear of the operator and a second side portion 134 having a second width “W2”. According to the embodiment shown in FIGS. 25 and 26, the first width W1 can be substantially equal to the second width “W2”. This particular design provides added coverage to the operator's ears and/or sides of their face, especially when compared with the prior art design, as illustrated in FIGS. 1-3. According to the embodiment shown in FIGS. 27 and 28, the second width “W2” can be less than the first width “W1”, wherein the second width “W2” is configured to enable air movement about the operator's face. This second width “W2” can also be designed so as to leave a portion of the operator's ear uncovered so that the operator's hearing is not impaired. The welder's dominant hand, i.e., whether the welder is right-handed or left-handed, will determine which side, i.e. the right side or left side of the welding hood 110, has the greater width W1. It can be appreciated that any of the side attachments or bottom attachments discussed above in regard to FIGS. 16-19 can be added to the welding hood 110 of FIGS. 25-28, thus providing additional protection and/or customization of the welding hood 110, as needed. It can also be appreciated that the welding hood of FIGS. 20A-28 can have similar sizes and dimensions as discussed above in regards to FIGS. 1-19.

As can be seen from the foregoing description and drawings, the welding hood of the present invention overcomes many of the deficiencies of the prior art by providing numerous safety features for the eyes, ears, and neck of the operator while also increasing the comfort to the operator. The welding hood can also be readily customized to meet a particular need of the operator and/or for use within a particular working environment.

While the disclosure has been described as having exemplary designs, the present disclosure can be further modified within the spirit and scope of this disclosure. This application is, therefore, intended to cover any variations, uses, or adaptations of the disclosure using its general principles. Further, this application is intended to cover such departures from the present disclosure as come within known or customary practice in the art to which this disclosure pertains and which fall within the limits of the appended claims.

The invention claimed is:

1. A welding hood for protecting an operator's face comprising:
a face protective shield having a rearward side configured for receiving the operator's face; a viewing opening extending through the shield;
an eye box located adjacent the viewing opening, said eye box having at least one wall framing at least a portion of the viewing opening and extending inwardly from the rearward side of the shield;
a lens configured for blocking the viewing opening; and
a light mounted to the face protective shield wherein the light shines outward from a front side of the face protective shield.

2. The welding hood of claim 1, wherein the light is removably located within a light holder, said light holder secured to the face protective shield, and wherein the light can be removed from the rearward side of the face protective shield.

3. The welding hood of claim 1, wherein the light comprises an LED light.

4. The welding hood of claim 1, wherein the eye box comprises a plurality of walls that are configured to fit flush against the operator's face and surround the operator's eyes to reduce the amount of sparks, dirt, and/or debris from coming into contact with the operator's eyes.

5. The welding hood of claim 1, wherein the lens is removably located within a holding member capable of pivoting between an open and closed position about the viewing opening and is formed from a light-filtering material for filtering light passing through the opening toward the rearward side of the hood.

6. The welding hood of claim 1, comprising an adjustable strap for securing the welding hood to an operator's head.

7. The welding hood of claim 1, including a fan mounted to the face protective shield.

8. The welding hood of claim 1, including a clear protective member secured to the eye box at a location adjacent to the viewing opening.

9. The welding hood of claim 1, wherein the face protective shield includes a first side portion having a first width configured for extending over at least a portion of the ear of the operator and a second side portion having a second width.

10. The welding hood of claim 9, wherein the second width of the second side portion is substantially equal to the first width of the first side portion so that both the first side portion and the second side portion extend over the ears of the operator.

11. The welding hood of claim 9, wherein the second width of the second side portion is less than the first width of the first side portion, wherein the second width is configured to enable air movement about the operator's face.

12. A welding hood for protecting an operator's face comprising:
a face protective shield having a rearward side configured for receiving the operator's face;
a first side portion having a first width configured for extending over at least a portion of the ear of the operator and a second side portion having a second width;
a viewing opening extending through the shield;
a lens located within the viewing opening;
a fan mounted to the face protective shield, said fan configured for supplying air to the operator's face; and
at least one strap for securing the welding hood to the operator's head.

13. The welding hood of claim 12, wherein the fan is mounted to the rearward side of the face protective shield.

14. The welding hood of claim 12, including a light mounted to the face protective shield.

15. The welding hood of claim 12, including an eye box located adjacent the viewing opening, said eye box having at least one wall framing at least a portion of the viewing opening and extending inwardly from the rearward side of the shield, wherein the at least one wall is configured for blocking at least one of reflective arc flashes, sunlight, and shop-lighting from entering the operator's field of view during welding.

16. The welding hood of claim 15, wherein the eye box includes a clear protective member mounted therein configured to provide eye protection to the operator.

17. The welding hood of claim 12, wherein the second width of the second side portion is less than the first width of the first side portion, wherein the second width is configured to enable air movement about the operator's face.

18. A welding hood for protecting an operator's face comprising:
a face protective shield having a rearward side configured for receiving the operator's face;
a viewing opening extending through the shield, said viewing opening located in a position in front of the eyes of the operator while the hood is fixed to the operator's head;
an eye box located adjacent the viewing opening, said eye box having at least one wall framing at least a portion of the viewing opening and extending inwardly from the rearward side of the shield, wherein the walls are configured for blocking at least one of reflective arc flashes, sunlight, and shop-lighting from entering the operator's field of view during welding;
a protective member secured to the eye box and located adjacent the viewing opening at a position in front of the eyes of the operator while the hood is fixed to the operator's eyes;
a holding member pivotally secured to a front side of the face protective shield, said holding member configured for holding a lens at a location adjacent to the viewing opening;
a fan secured to the rearward side of the face protective shield;
a light mounted to the face protective shield wherein the light shines outward from a front side of the face protective shield.

19. The welding hood of claim 18, including at least one strap for securing the welding hood to the operator's head.

20. The welding hood of claim 18, wherein the face protective shield includes a first side portion having a first width configured for extending over at least a portion of the ear of the operator and a second side portion having a second width that is less than the first width of the first side portion, wherein the second width is configured to enable air movement about the operator's face.
